(19) United States
(12) Patent Application Publication
(10) Pub. No.: US 2009/0113591 A1
(43) Pub. Date: May 7, 2009

(54) Limb Protector

(76) Inventors: Henry Deutsch, Sandy, UT (US); Timothy R. Hartigan, Evanston, IL (US); Matthew L. Jiminez, Evanston, IL (US)

Correspondence Address: HOOKER & HABIB, P.C., 100 CHESTNUT STREET, SUITE 304, HARRISBURG, PA, 17101, US

(21) Appl. No.: 12/117,518

(22) Filed: May 8, 2008

Related U.S. Application Data
(60) Provisional application No. 60/916,856, filed on May 9, 2007.

(51) Int. Cl. A41D 13/08; A41D 13/00; A41D 19/01; A41D 19/00; A61F 5/00

(52) U.S. Cl. 2/16; 2/22; 2/158; 2/159; 602/3

(57) ABSTRACT

A limb protector for protecting a limb of a wearer from contact with water or other contaminant has a tubular member made from ultrathin plastic film. The ultrathin film enables the material around the opening to form a watertight seal around the limb without forming a venous tourniquet.

### FIELD OF THE INVENTION

The invention relates to a protective cover for protecting a limb of a wearer from contact with water or other contaminant.

### DESCRIPTION OF THE PRIOR ART

Limb protectors are used to cover arms or legs and prevent the arm or leg from getting wet, or in protecting casts or splints applied to the arm or leg from being softened by contact with moisture. In addition to preventing water or other contaminants such as paints or spray finishes from contacting the arms or legs, such protectors are also useful in retaining medications or other dressings applied to extremities or isolating injured or pathologic areas of the extremities.

Conventional limb protectors are waterproof tubular bags having an opening at one end of the bag. The limb is inserted through the opening and into the bag. The opening is then sealed to prevent fluids from entering the bag. The protectors are often used once and then disposed of after use.

In one type of conventional protector the opening is sealed by stretching an elastic or rubber band around the bag adjacent the opening. The band must be provided with the bag, and it can be cumbersome for the user to apply the band without assistance.

In another type of conventional protector the opening is formed in a tubular elastomeric cuff or sleeve attached or formed adjacent the bag. The sleeve elastically expands to receive the limb and then squeezes the limb to form a watertight seal. The elastomeric material must be attached to the bag, increasing cost and making the protector less economical for one-time use.

Yet another conventional limb protector is a tubular member made from plastic, with an opening at one end to insert the limb. The tubular member is made from thick polyurethane film having a film thickness of from 0.005 inches to 0.020 inches. The film around the opening elastically stretches to receive the limb and fits snugly around the limb to be protected.

The film pressure around the opening of the tubular member compresses the skin and the underlying veins, effectively forming a venous tourniquet. The film pressure is not high enough to compress the deeper arteries. Blood flows into the limb through the arteries but cannot flow out because of the tourniquet formed at the member opening. This may cause significant swelling or may injure the limb itself.

Thus there is a need for an improved limb protector that is easy to apply and economical for even one-time use, and is not likely to form a venous tourniquet.

### SUMMARY OF THE INVENTION

The present invention is an improved limb protector that is easy to apply and economical for even one-time use, and is not likely to form a venous tourniquet.

A limb protector in accordance with the present invention is a tubular member formed from an ultrathin plastic film. The applicants discovered the surprising and unexpected result that a limb protector made from ultrathin plastic film can be durable, form a satisfactory seal around the limb, and form the seal without forming a venous tourniquet.

The tubular member has an opening to receive a limb. The opening has a cross-sectional area less than the maximum cross-sectional area of the limb being received through the opening. Inserting the limb through the opening forms a waterproof seal around the limb.

The material around the opening recovers and becomes stretched against the limb to form the watertight seal. However, the film is an ultrathin film, not a thick film as used in conventional limb protectors, and the compressive force applied to the limb at the opening by the ultrathin film is sufficiently high to form a satisfactory seal but not so high as to form a venous tourniquet.

The improved limb protector of the present invention is easy to apply. Inserting the limb into the opening automatically forms the seal, without the need of an elastic band or other assistance. The force required to deform the opening is low because of the use of ultrathin film, and does not noticeably impede or hinder entry of the limb into the bag. The compressive force applied to the limb by the ultrathin film at the opening is low and does not prevent the veins from flowing blood out of the limb protector.

The tubular member is formed from an ultrathin film of flexible planar material. Thermoplastic polyurethane (“TPU”) is a preferred material for forming the tubular member. TPU is commercially available from many plastic vendors and can be obtained as a clear, waterproof film. The film is relatively inexpensive and limb protectors can be made economically for one-time use. Latex, PVC, and other film materials are known and can be adapted for use with the present invention.

In preferred embodiments of the limb protector, the limb protector is made from ultrathin polyurethane film, preferably a film 0.005 millimeters thick. This film thickness has satisfactory strength, elasticity, and recovery characteristics. The ultrathin film also forms a flexible tubular member that is easily folded for packaging and storage.

The film is cut to shape and a first side is folded over a second side along a fold line. The side edges are sealed together along their outer perimeters, preferably by RF welding. Heat sealing, adhesive bonding, blow molding, or other conventional sealing or manufacturing techniques could also be used to form the tubular member. The end of the tubular member opposite the opening can be formed as a mitten or glove if desired.

The opening is preferably formed in the fold line so that the material's plastic deformation takes place within the film and does not extend to the sealed edges of the tubular member. Different sized openings are formed in different embodiments, depending on whether the limb protector is for the arm or leg, and whether the limb protector is for adults, children, or infants. Embodiments designed for animal use are also within the intended scope of the invention.

Other objects and features of the invention will become apparent as the description proceeds, especially when taken in conjunction with the accompanying two drawing sheets illustrating two embodiments of the invention.

### BRIEF DESCRIPTION OF THE DRAWINGS

FIG. 1 is a front view of a preform for forming a first embodiment limb protector in accordance with the present invention;

FIG. 2 is a front view of the limb protector formed from the preform shown in FIG. 1;

FIG. 3 is a partial side view taken along line 3-3 of FIG. 2;

FIG. 4 is a representational view illustrating the relative cross sectional areas of the limb protector opening and the limb inserted through the opening; and

FIG. 5 is a front view of a second embodiment limb protector in accordance with the present invention.

### DESCRIPTION OF THE PREFERRED EMBODIMENTS

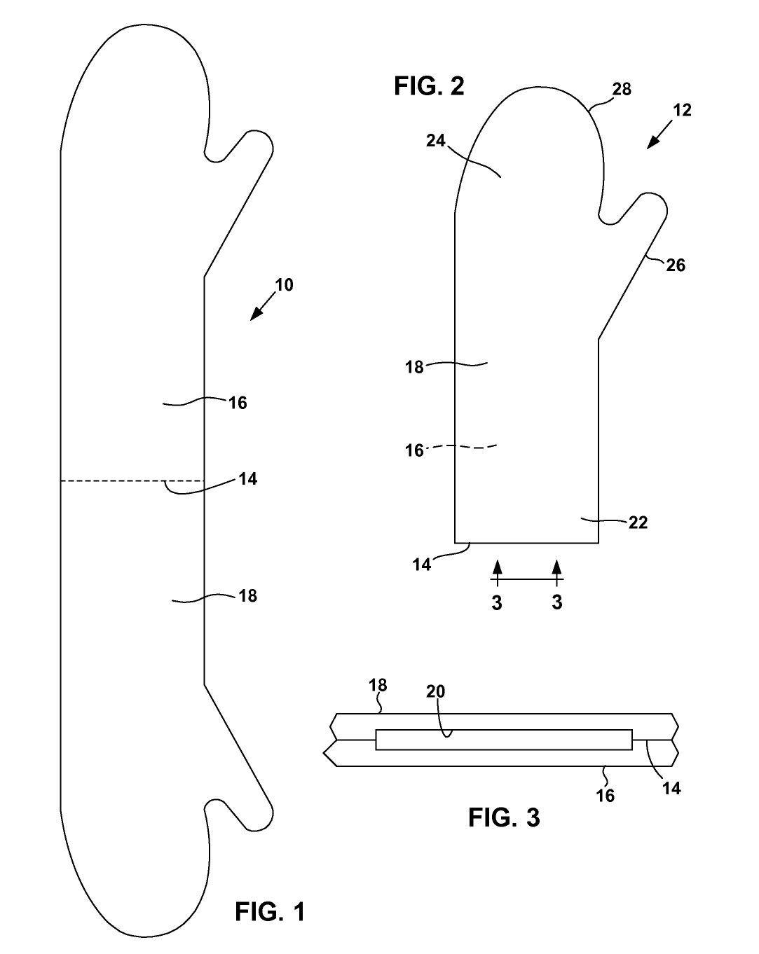

FIG. 1 illustrates a preform 10 for forming an arm protector 12 shown in FIGS. 2 and 3 in accordance with the present invention. Protector 12 is a tubular member intended to receive an arm of an adult wearer.

Preform 10 is cut from a ultrathin thermoplastic polyurethane film, preferably less than 0.01 millimeters thick. The illustrated embodiment is formed from thermoplastic polyurethane film having a film thickness of 0.005 millimeters. The preform 10 is folded over a fold line 14 that divides the preform into two like, opposite sides 16, 18. The perimeter edges of sides 16, 18 are sealed together by RF welding in a conventional manner to form a watertight seal between the edges.

An opening 20 (see FIG. 3) is cut into the fold line 14 to form an open end 22 of protector 12. The opposite closed end 24 of illustrated protector 12 is configured as a mitten having tubular thumb-receiving portion 26 and fingers-receiving portion 28. End 24 could also be otherwise shaped as is known in the art, and could be configured as a glove having a plurality of digit-receiving portions.

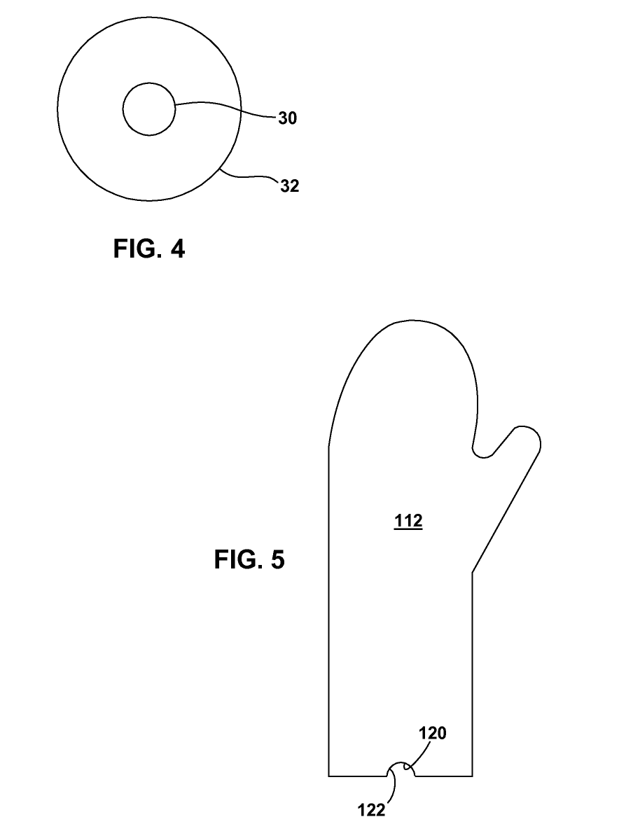

Opening 20 extends only partially along fold line 14 and defines an opening having a cross-sectional area represented by the area of circle 30 shown in FIG. 4. The cross-sectional area of the limb to be received through the opening 20 is represented by the area of circle 32. The area 30 of opening 20 is substantially less than the area 32 of the limb being received through the opening 20.

Area 30 is selected to cause plastic deformation of the material around opening 20 when the limb is inserted through the opening. Preferably opening 20 has a diameter of about 4 centimeters to receive an adult hand and arm and about 6 centimeters to receive an adult foot and leg. Opening sizes can be correspondingly reduced for use with children and infants. The cross-sectional area of the tubular member 12 away from opening 20 is sufficiently greater than area 32 to surround and protect the limb without otherwise necessarily touching the skin or a conventional cast or splint on the limb.

Inserting the arm into the tubular member 12 through opening 20 plastically deforms the tubular member 12 around the opening 20. After the arm has been inserted, recovery or spring back of the plastically-deformed material at the opening is resisted by the arm. The material facing the opening forms a watertight, linear seal at the material-limb interface.

The user can reuse arm protector 12. Removing the arm from the tubular member 12 may leave an enlarged opening 20 because of possible plastic deformation of the material about opening 20. The enlarged opening 20 has less cross-sectional area than the limb area 32 because the plastically deformed material retains some elasticity. The arm can be reinserted into tubular member 12 and the retained elasticity of the material enables a watertight seal to form again at the material-limb interface.

FIG. 5 illustrates a second embodiment limb protector 112 in accordance with the present invention. Limb protector 112 is otherwise identical to limb protector 12, but the opening 120 is formed along a half-moon cutout 122 formed in the sides of the protector 112, 114. The cutout 122 enables a user to quickly locate the opening 120 for use.

Other embodiment of the invention may have an additional opening to permit the limb protector to be placed on the arm above the hand or on the leg above the foot.

While we have illustrated and described preferred embodiments of our invention, it is understood that this is capable of modification, and we therefore do not wish to be limited to the precise details set forth, but desire to avail ourselves of such changes and alterations as fall within the purview of the following claims.

What we claim as our invention is:

1. A limb protector for protecting a limb of a wearer from contact with water or other contaminant, the limb protector comprising:
an elongated, generally tubular waterproof member formed from ultrathin plastic film;
the tubular member comprising opposite ends, one end having an opening to receive a limb, the other end being a closed end, the opening having a cross-sectional area sufficiently less than the maximum cross-sectional area of the limb being received through the opening wherein inserting the limb plastically deforms the material around the opening; and
wherein the recovery characteristic of the material enables the material around the opening to recover after plastic deformation and form a waterproof seal around the limb.

2. The limb protector of claim 1 wherein the plastic film is thermoplastic polyurethane.

3. The limb protector of claim 1 wherein the one end of the tubular member comprises a fold line, the opening in the fold line.

4. The limb protector of claim 3 wherein the tubular member comprises a first side folded over a second side at the fold line, the first side sealed to the second side by at least one of the following: RF welding, heat sealing, and adhesive bonding.

5. The limb protector of claim 1 wherein the opening is configured to form a linear seal against the limb.

6. The limb protector of claim 1 wherein the other end of the tubular member is configured as a glove or mitten comprising a tubular portion configured to receive at least one separate digit of the limb.

7. The limb protector of claim 1 wherein the tubular member comprises a film, a fold line in the film dividing the film into opposite sides, one side permanently attached to the other side and forming the entire tubular member.

8. The limb protector of claim 1 wherein the material around the opening retains elasticity after plastic deformation sufficient to enable reuse of the limb protector.

9. The limb protector of claim 1 wherein the film has a thickness of about 0.005 millimeters.

10. A method of protecting a human or other mammal limb from contact with water or other contaminant, the method comprising the steps of:
(A) providing a tubular member made from ultrathin film having an opening at one end of the member;
(B) inserting the limb into the tubular member through the opening;
(C) deforming the ultrathin film at the opening by inserting the limb into the tubular member;
(D) recovering sufficient deformation of the ultrathin film at the opening to form a waterproof seal around the limb without forming a venous tourniquet.

11. The method of claim 10 herein step (A) comprises the step of:
(E) forming the tubular member from thermoplastic polyurethane.

12. The method of claim 10 herein step (A) comprises the step of:
(E) folding a planar material along a fold line.

13. The method of claim 12 wherein step (A) comprises the step of:
(F) forming the opening in the fold line.

14. The method of claim 12 wherein step (A) comprises the step of:
(G) sealing edges of the folded material to form the tubular member.

15. The method of claim 10 wherein step (C) comprises the step of:
(E) inserting a limb portion having a cross-sectional area greater than the cross sectional area of the opening through the opening.

16. The method of claim 10 wherein step (D) comprises the step of:
(E) retaining the limb within the opening and thereby resisting springback of the tubular member at the opening.

17. The method of claim 10 wherein the opening in step (A) has a first cross-section area, the method comprising the steps of:
(E) removing the limb from the tubular member;
(F) re-inserting the limb into the tubular; and
(G) elastically deforming the tubular member around the opening while re-inserting the limb into the tubular member, the elastic deformation sufficient to form a waterproof seal around the limb after re-inserting the limb.

18. The method of claim 10 wherein the ultrathin film has a thickness of about 0.005 millimeters.
